(19) United States
(12) Patent Application Publication
(10) Pub. No.: US 2021/0299309 A1
(43) Pub. Date: Sep. 30, 2021

(54) TENT WITH ANTIMICROBIAL TREATMENT APPLIED THERETO AND METHOD OF IMPARTING ANTIMICROBIAL PROPERTIES TO THE TENT

(71) Applicant: Parasol Medical, LLC, Buffalo Grove, IL (US)

(72) Inventor: Patrick E. Eddy, Allendale, MI (US)

(73) Assignee: Parasol Medical, LLC, Buffalo Grove, IL (US)

(21) Appl. No.: 17/205,630

(22) Filed: Mar. 18, 2021

Related U.S. Application Data
(60) Provisional application No. 63/002,731, filed on Mar. 31, 2020.

(51) Int. Cl. A61L 2/232; A61L 2/18; A61L 31/10; A61L 31/08

(52) U.S. Cl. CPC A61L 2/232; A61L 2/186; A61L 2101/46; A61L 31/088; A61L 31/10

(57) ABSTRACT

A tent comprising an exposed surface and an antimicrobial substance applied to the exposed surface. The tent can further include an interior. The exposed surface can be disposed within the interior. The tent can include a fabric. The fabric can provide the exposed surface. The tent can be a medical center. The antimicrobial substance can be a silane quaternary ammonium ion or salt thereof. The silane quaternary ammonium ion can be one or more of: 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, and 3-(trihydroxysilyl)propyldimethyloctadecyl ammonium chloride.

### CROSS-REFERENCE TO RELATED APPLICATION

This application claims priority under 35 U.S.C. § 119(e) and the benefit of prior-filed Provisional Application No. 63/002,731, entitled TENT WITH ANTIMICROBIAL TREATMENT APPLIED THEREFOR AND METHOD OF IMPARTING ANTIMICROBIAL PROPERTIES TO THE TENT, filed on 31 Mar. 2020, the entirety of which is incorporated herein by reference.

### BACKGROUND

A tent is sometimes utilized as temporary shelter. More than one person can sometimes occupy the tent simultaneously. In some instances, the tent is utilized as a medical center where health care providers provide medical treatment to other people that require medical treatment.

However, there is a problem in that the tent can harbor pathogens on an exposed surface and, thereby, serve as a vehicle to transmit the pathogens to other people. In instances where the tent is being utilized as a medical center, the exposed surface of the tent can transfer pathogens between those receiving medical treatment and between those providing medical care.

### BRIEF SUMMARY OF THE INVENTION

The present disclosure solves that problem by treating the exposed surface of the tent with a solution including an antimicrobial substance, such as a silane quaternary ammonium ion or salt thereof. The solution dries leaving the antimicrobial substance applied to (i.e., disposed on) the exposed surface of the tent. In the event that pathogens are transferred to the exposed surface of the tent to which the antimicrobial substance is additionally applied, the antimicrobial substance can inactivate the pathogens and, thereby, reduce the likelihood that the pathogens are subsequently transferred from the exposed surface of the tent to another person within the tent.

In a first aspect of the present disclosure, a tent comprises an exposed surface and an antimicrobial substance applied to the exposed surface. In embodiments, the tent further comprises an interior, and the exposed surface is disposed within the interior. In embodiments, the tent further comprises a fabric, and the fabric comprises the exposed surface. In embodiments, the tent is a medical center. In embodiments, the antimicrobial substance is a silane quaternary ammonium ion or salt thereof applied to the exposed surface. In embodiments, the silane quaternary ammonium ion or salt thereof is one or more of: 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, and 3-(trihydroxysilyl)propyldimethyloctadecyl ammonium chloride. In embodiments, the tent further comprises hydrogen peroxide applied to the exposed surface. In embodiments, the tent further comprises hypochlorous acid applied to the exposed surface.

In a second aspect of the present disclosure, a method of imparting antimicrobial properties to a tent comprises: applying, to an exposed surface of a tent, a solution comprising an antimicrobial substance. In embodiments, the tent comprises an interior, and the exposed surface is disposed within the interior. In embodiments, the tent comprises a fabric, and the fabric comprises the exposed surface. In embodiments, the tent is a medical center. In embodiments, applying the solution comprises spraying the solution onto the exposed surface of the tent. In embodiments, spraying comprises spraying the solution with an electrostatic sprayer. In embodiments, the antimicrobial substance comprises a silane quaternary ammonium ion or salt thereof. In embodiments, the solution further comprises a solvent. In embodiments, the silane quaternary ammonium ion or salt thereof is between 0.1 percent and 10 percent by weight of the solution. In embodiments, the solvent is isopropyl alcohol. In embodiments, isopropyl alcohol is between 30 percent to 90 percent by weight of the solution.

In embodiments, the solution further comprises hydrogen peroxide. In embodiments, the solution further comprises hypochlorous acid. In embodiments, the silane quaternary ammonium ion or salt thereof is one or more of: 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, and 3-(trihydroxysilyl)propyldimethyloctadecyl ammonium chloride.

These and other features, advantages, and objects of the present invention will be further understood and appreciated by those skilled in the art by reference to the following specification, claims, and appended drawings.

### BRIEF DESCRIPTION OF THE DRAWINGS

FIG. 1 is a perspective view of a tent, specifically a medical tent, illustrating an exterior with an entrance into an interior, and the exterior having an exposed surface with an antimicrobial substance applied thereto;

FIG. 2 is a perspective view of the interior of the tent of FIG. 1, illustrating the interior having an exposed surface with the antimicrobial substance applied thereto, and a person applying, via spraying, the antimicrobial substance onto the exposed surface of the interior with an electrostatic sprayer; and

FIG. 3 is a flow chart of a method of imparting antimicrobial properties to a tent illustrating the step of applying, to the exposed surface of the tent, a solution comprising the antimicrobial substance.

### DETAILED DESCRIPTION

For purposes of description herein, it is to be understood that the specific devices and processes illustrated in the attached drawings, and described in the following specification are simply exemplary embodiments of the inventive concepts defined in the appended claims. Hence, physical characteristics relating to the embodiments disclosed herein are not to be considered as limiting, unless the claims expressly state otherwise.

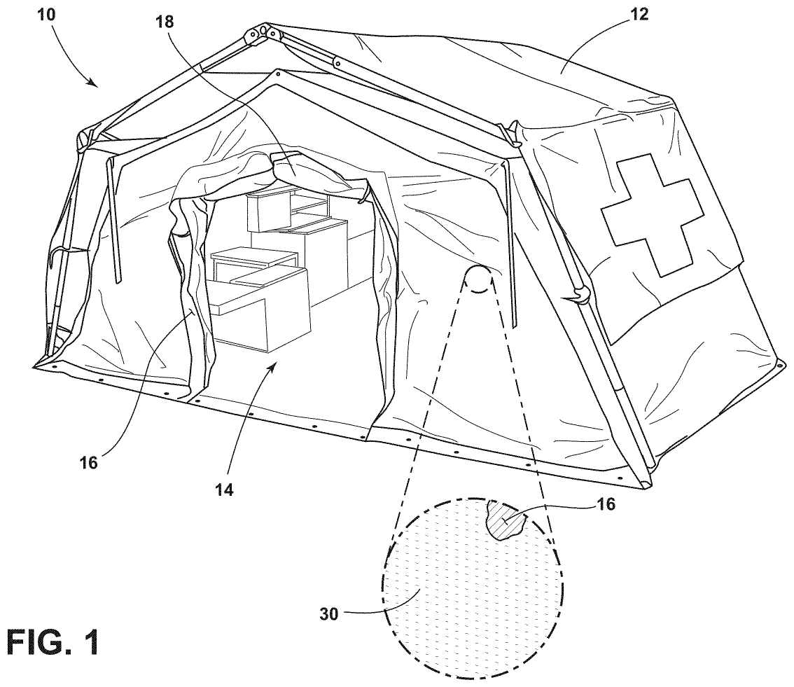

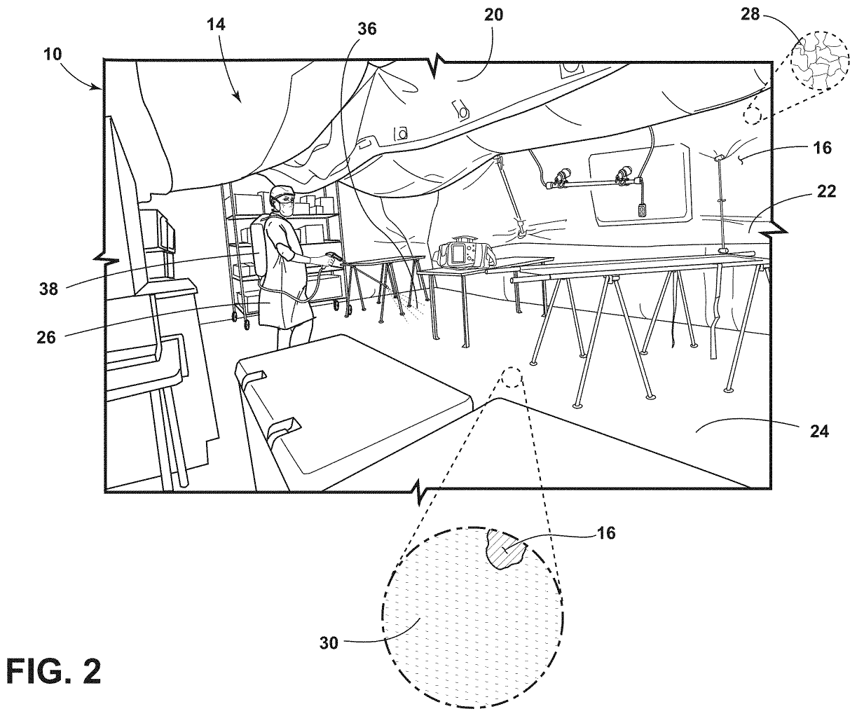

Referring now to FIGS. 1 and 2, a tent 10 is illustrated. The tent 10 includes an exterior 12 and an interior 14. The tent 10 further includes exposed surfaces 16, at both the exterior 12 and interior 14. In other words, the exposed surface 16 can be disposed within the interior 14. The exposed surface 16 can be disposed at the exterior 12, or both the within the interior 14 and the exterior 12. As another example, the exposed surface 16 can be at the exterior 12 near an entrance 18 into the interior 14. As another example, the exposed surface 16 can be disposed at a ceiling 20, a sidewall 22, or a floor 24 within the interior 14. These examples are not exclusive. An “exposed surface” is any surface that is open to the environment and capable of being contacted by a person 26. The tent 10 can further include a fabric 28, which can be woven or non-woven, and the fabric 28 can provide the exposed surface 16. In embodiments, the tent 10 is a medical center. A “medical center” is a place where medical care is provided. In other embodiments, the tent 10 is a temporary shelter used for camping, hiking, and other similar activities.

The tent 10 further comprises an antimicrobial substance 30 applied to the exposed surface 16. Stated another way, the antimicrobial substance 30 is disposed on the exposed surface 16. The antimicrobial substance 30 can be a coating over the exposed surface 16. In some embodiments, the antimicrobial substance 30 is bonded, either chemically or physically, to the exposed surface 16. In embodiments, the tent 10 further comprises hydrogen peroxide applied to the exposed surface 16, either simultaneously with the application of the antimicrobial substance 30 or before (or after) the application of the antimicrobial substance 30 to the exposed surface 16. In embodiments, the tent 10 further comprises hypochlorous acid applied to the exposed surface 16, either simultaneously with the application of the antimicrobial substance 30 or before (or after) the application of the antimicrobial substance 30 to the exposed surface 16.

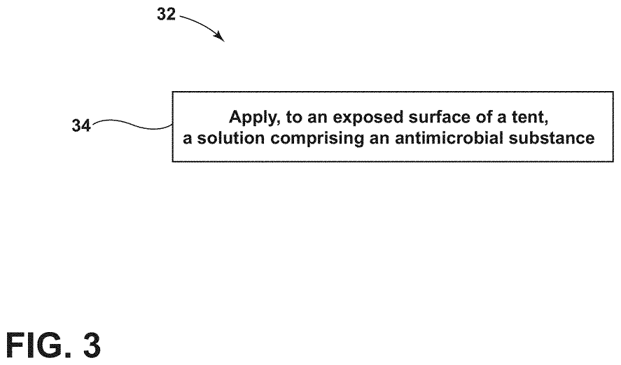

Referring now to FIG. 3, a method 32 of imparting antimicrobial properties to the tent 10 comprises, at a step 34, applying, to an exposed surface 16 of a tent 10, a solution comprising the antimicrobial substance 30. In embodiments, the step 34 of applying the solution comprises spraying 36 the solution onto the exposed surface 16 of the tent 10.

In embodiments, spraying 36 comprises spraying the solution with an electrostatic sprayer 38. In other embodiments, the step 34 of applying the solution comprises wiping the exposed surface 16 with a wipe that includes the solution. In other embodiments, the step 34 of applying the solution comprises dipping the exposed surface 16 into the solution. In embodiments, the solution further comprises a solvent and, after the step 34 of applying the solution, the solvent evaporates leaving the antimicrobial substance disposed on the exposed surface 16.

In embodiments, the solution further comprises hydrogen peroxide. In embodiments, the hydrogen peroxide is 0.05 percent by weight to 7.5 percent by weight of the solution.

In embodiments, the solution further comprises hypochlorous acid. In embodiments, the solution comprises hypochlorous acid, a silane quaternary ammonium ion or salt thereof, and water. In embodiments, the hypochlorous acid exists in the solution at a concentration of 0.1 to 1000 ppm. In embodiments, the hypochlorous acid exists in the solution at a concentration of 0.1 to 200 ppm. In embodiments, the hypochlorous acid exists in the solution at a concentration of 200 to 1000 ppm.

Because the antimicrobial substance 30 is disposed on the exposed surface 16, the antimicrobial substance 30 can inactivate a pathogen that deposits on the exposed surface 16. Inactivation includes killing the pathogen or slowing propagation of the pathogen. In some instances, applying the antimicrobial substance 30 onto the exposed surface 16 can inactivate pathogens already present on the exposed surface 16 and thus can be utilized as a mitigation measure.

In embodiments, the antimicrobial substance is a silane quaternary ammonium ion or salt thereof. In embodiments, the silane quaternary ammonium ion or salt thereof is one or more of: 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, and 3-(trihydroxysilyl)propyldimethyloctadecyl ammonium chloride. In embodiments, the silane quaternary ammonium ion or salt thereof is between 0.1 percent and 10 percent by weight of the solution. The silane quaternary ammonium ion or salt thereof chemically bonds to the exposed surface 16 to which the solution is applied, thus providing a long-lasting antimicrobial effect. When the solution includes both hydrogen peroxide or hypochlorous acid and the silane quaternary ammonium ion or salt thereof, the hydrogen peroxide or hypochlorous acid provides a relatively instant antimicrobial effect to destroy microbes present on the exposed surface 16, and the silane quaternary ammonium ion or salt thereof chemically bonds to the exposed surface 16 to provide the residual antimicrobial effect to destroy microbes that become disposed on the exposed surface 16.

In embodiments, the solvent is isopropyl alcohol. In embodiments, isopropyl alcohol is between 30 percent to 90 percent by weight of the solution. In embodiments, isopropyl alcohol is 30 percent, 35 percent, 40 percent, 45 percent, 50 percent, 55 percent, 60 percent, 65 percent, 70 percent, 75 percent, 80 percent, 85 percent, 90 percent by weight of the solution, or within any range bounded by any two of those values (e.g., 55 percent to 65 percent by weight, 50 percent to 60 percent by weight, 60 percent to 70 percent by weight, etc.). In embodiments, the solution comprises 55 percent to 65 percent by weight isopropyl alcohol and 0.1 percent to 10 percent by weight the silane quaternary ammonium ion or salt thereof, and the silane quaternary ammonium ion or salt thereof is one or more of: 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, and 3-(trihydroxysilyl)propyldimethyloctadecyl ammonium chloride. An example solution comprises (by weight) 60.0 percent isopropyl alcohol, 2.02 percent 3-(trimethoxysilyl)propyldimethyloctadecyl ammonium chloride, and 34.19 percent deionized water.

It is to be understood that variations and modifications can be made on the aforementioned structure without departing from the concepts of the present invention, and further, it is to be understood that such concepts are intended to be covered by the following claims unless these claims by their language expressly state otherwise.

What is claimed is:

1. A tent comprising an exposed surface and an antimicrobial substance applied to the exposed surface.

2. The tent of claim 1 further comprising an interior, wherein
the exposed surface is disposed within the interior.

3. The tent of claim 1 further comprising a fabric, wherein
the fabric comprises the exposed surface.

4. The tent of claim 1, wherein
the tent is a medical center.

5. The tent of claim 1, wherein
the antimicrobial substance is a silane quaternary ammonium ion or salt thereof.

6. The tent of claim 5, wherein
the silane quaternary ammonium ion or salt thereof is one or more of:
3-(trimethoxysilyl)propyldimethyloctadecyl ammonium ion,
3-(trimethoxysilyl)propyldimethyloctadecyl ammonium chloride,
3-(trihydroxysilyl)propyldimethyloctadecyl ammonium ion, and
3-(trihydroxysilyl)propyldimethyloctadecyl ammonium chloride.

7. The tent of claim 5 further comprising:
hydrogen peroxide applied to the exposed surface.

8. The tent of claim 5 further comprising:
hypochlorous acid applied to the exposed surface.

9. A method of imparting antimicrobial properties to a tent comprising:
applying, to an exposed surface of a tent, a solution comprising an antimicrobial substance.

10. The method of claim 9, wherein
the tent comprises an interior; and
the exposed surface is disposed within the interior.

11. The method of claim 9, wherein
the tent comprises a fabric; and
the fabric comprises the exposed surface.

12. The method of claim 9, wherein
the tent is a medical center.

13. The method of claim 9, wherein
applying the solution comprises spraying the solution onto the exposed surface of the tent.

14. The method of claim 13, wherein
spraying comprises spraying the solution with an electrostatic sprayer.

15. The method of claim 9, wherein
the antimicrobial substance comprises a silane quaternary ammonium ion or salt thereof; and
the solution further comprises a solvent.

16. The method of claim 15, wherein
the silane quaternary ammonium ion or salt thereof is between 0.1 percent and 10 percent by weight of the solution.

17. The method of claim 15, wherein
the solvent is isopropyl alcohol; and
the isopropyl alcohol is between 30 percent to 90 percent by weight of the solution.

18. The method of claim 9, wherein
the solution further comprises hydrogen peroxide; and
the antimicrobial substance comprises a silane quaternary ammonium ion or salt thereof.

19. The method of claim 9, wherein
the solution further comprises hypochlorous acid; and
the antimicrobial substance comprises a silane quaternary ammonium ion or salt thereof.

20. The method of claim 15, wherein
the silane quaternary ammonium ion or salt thereof is one or more of:
3-(trimethoxysilyl)propyldimethyloctadecyl ammonium ion,
3-(trimethoxysilyl)propyldimethyloctadecyl ammonium chloride,
3-(trihydroxysilyl)propyldimethyloctadecyl ammonium ion, and
3-(trihydroxysilyl)propyldimethyloctadecyl ammonium chloride.
